SEQUENCE LISTING (9 sequences)

SEQ ID NO: 1 - DNA, 11 bp, Artificial Sequence
  Example sequence to show the method for detecting the existence of methylated sites in a nucleotide-containing compound (NCC) - original template
tactaaacgat

SEQ ID NO: 2 - DNA, 11 bp, Artificial Sequence
  Example sequence to show the method for detecting the existence of methylated sites in a NCC without methylated cytosine - original template
atcgtttagta

SEQ ID NO: 3 - DNA, 11 bp, Artificial Sequence
  Modified NCC from SEQ ID No. 1 showing non-methylated cytosine replaced with uracil
tantaaacgat

SEQ ID NO: 4 - DNA, 11 bp, Artificial Sequence
  Modified NCC from PCR product of SEQ ID No. 1 - showing replacement of cytosine with uracil
tantaaangat

SEQ ID NO: 5 - DNA, 11 bp, Artificial Sequence
  Modified NCC by converting cytosine to uracil from SEQ ID NO. 2
atugtttagta

SEQ ID NO: 6 - DNA, 11 bp, Artificial Sequence
  modified NCC which is the PCR product of SEQ ID NO. 3 with replacement of uracil with thymine
tattaaacgat

SEQ ID NO: 7 - DNA, 11 bp, Artificial Sequence
  Complementary sequence of SEQ ID NO. 6 (5′ to 3′)
atcgtttaata

SEQ ID NO: 8 - DNA, 11 bp, Artificial Sequence
  PCR product of SEQ ID NO. 4 with replacement of uracil with thymine
tattaaatgat

SEQ ID NO: 9 - DNA, 11 bp, Artificial Sequence
  Complementary sequence of SEQ ID NO. 8 (5′ to 3′)
atcatttaata

(19) United States
(12) Patent Application Publication
(10) Pub. No.: US 2004/0048276 A1
(43) Pub. Date: Mar. 11, 2004

(54) System and method for determining the presence of methylated cytosines in polynucleotides

(76) Inventors: Zhaowei Liu, Port Matilda, PA (US); Qingbo Li, State College, PA (US)

Correspondence Address: JONES DAY, 222 EAST 41ST STREET, NEW YORK, NY, 10017, US

(21) Appl. No.: 10/411,103

(22) Filed: Apr. 11, 2003

Related U.S. Application Data
(63) Continuation of application No. PCT/US01/31665, filed on Oct. 10, 2001.
(60) Provisional application No. 60/239,119, filed on Oct. 11, 2000.

(51) Int. Cl. C12Q 1/68; C12P 19/34

(52) U.S. Cl. 435/6; 435/91.2

(57) ABSTRACT

The present invention relates to a method for determining the presence of a methylated cytosine in a first sample comprising a first nucleotide containing compound. The first sample and a reference NCC are subjected to electrophoresis in the presence of at least one intercalating dye. During electrophoresis the temperature of the first NCC and the reference NCC is changed by an amount sufficient to change an electrophoretic mobility of at least one of the first or reference NCCs. Fluorescence intensity data are obtained. The fluorescence intensity data are indicative of the presence of the first and reference NCCs.

### RELATED APPLICATIONS

The present application is a continuation of international application no. PCT/US01/31665, filed Oct. 10, 2001 and U.S. Provisional Application No. 60/239,119, filed Oct. 11, 2000.

### FIELD OF THE INVENTION

The invention relates to a system and method for separating materials having temperature-dependent electrokinetic mobilities. More particularly, the invention relates to time-dependent temperature gradient electrokinetic separation of materials including DNA fragments.

### BACKGROUND

Methylation of cytosine residues has received enormous attention in recent years because of its important roles in transcription inactivation and in cancer development. Efficient methods are required in order to accomplish the daunting task of mapping methylated cytosines in human DNA because existing DNA sequencing technologies can not directly detect such methylation sites. In other words, the existing DNA sequencing chemistry cannot distinguish between methylated and un-methylated cytosines.

Currently, two major techniques are applied to map methylated cytosine (mC) sites in the genome. Southern analysis of DNA fragments digested by methylation-sensitive and -insensitive restriction enzymes generates information of mC only on the restriction sites along the genome. Differential cytosine modification by sodium bisulfite treatment separates mCs from non-methylated mCs and maps them to specific DNA sites. U.S. Pat. No. 6,017,704 to Herman et al., which is incorporated herein to the extent necessary to understand the present invention, discloses a methylation specific PCR method that chemically modifies both methylated and non-methylated DNA. After modification, only the methylated sites are revealed as different bases between the fragments generated from methylated and non-methylated fragments. However, only limited mC sites can be surveyed by this procedure. It is necessary to develop a high-throughput technique for detecting mCs over larger regions of DNA.

### SUMMARY OF THE INVENTION

One embodiment of the present invention relates to a method for detecting the existence of methylated sites in a nucleotide-containing compound NCC, which is preferably a polynucleotide, such as a single or double strand of DNA. The method preferably comprises contacting the NCC with a second compound to thereby provide a modified NCC, wherein non-methylated cytosines are replaced with another base, which is preferably uracil. The modified NCC is amplified by a PCR reaction to provide PCR products. The PCR products are preferably combined with PCR products resulting from the amplification of a reference NCC, which is essentially free of methylated cytosines. The combined products are denatured and re-annealed to provide homoduplexes and heterodupexes if the NCC included at least one methylated cytosine.

The combined PCR products are subjected to temperature gradient electrophoresis, preferably in one or more capillaries. The PCR products are irradiated with light to provide a spectroscopic signal, which is preferably a fluorescence or absorbance signal. The spectroscopic signal is converted to data, which are indicative of the presence of methylated cytosines present in the NCC. The data are preferably an electropherogram that includes peaks indicative of the presence of the at least partially separated PCR products. Methylated cytosines are preferably indicated by the presence of additional or broadened peaks compared to an electropherogram obtained when the NCC had no methylated cytosines.

Another embodiment of the present invention relates to a temperature gradient electrophoresis-based method for generating data indicative of the presence of one or more methylated cytosines in a sample comprising a first nucleotide containing compound (NCC) having non-methylated cytosines. The method comprises contacting the first NCC with a first compound to thereby provide a modified NCC wherein non-methylated cytosines of the first NCC are replaced with a different base. The modified NCC is amplified to obtain first PCR products. The first PCR products and a reference NCC are subjected to temperature gradient electrophoresis. The first PCR products and reference NCC are irradiated with light to thereby generate a spectroscopic signal. The spectroscopic signal is converted into data indicative of the presence of the one or more methylated cytosines in the first NCC.

Yet another embodiment of the invention relates to a temperature gradient electrophoresis-based method for generating data indicative of the presence of one or more methylated cytosines in a sample comprising a first nucleotide containing compound (NCC). The method comprises obtaining first PCR products formed by: (a1) contacting the first NCC with a first compound to thereby provide a modified NCC in which non-methylated cytosines are replaced with a different base, and (a2) amplifying the modified NCC to obtain said first PCR products. The first PCR products and a reference NCC are subjected to temperature gradient electrophoresis. The first PCR products and reference NCC are irradiated with light to thereby generate a spectroscopic signal. The spectroscopic signal is converted into data indicative of the presence of the one or more methylated cytosines in the first NCC.

### BRIEF DESCRIPTION OF THE DRAWINGS

The present invention is described in detail below with reference to the drawings in which:

FIG. 1 shows a flow chart of steps to prepare a nucleotide containing compound for obtaining data indicative of the presence of methylated cytosine;

FIG. 2 shows the hybridization of nucleotide containing compounds obtained by following the steps of the flow chart of FIG. 1;

FIG. 3a shows an embodiment of a methylated cytosine detection device having a gas cooled portion in accordance with the present invention;

FIG. 3b shows an embodiment of a methylated cytosine detection device having a thermoelectrically cooled portion in accordance with the present invention;

FIG. 3c shows an embodiment of a methylated cytosine detection device having a liquid cooled portion in accordance with the present invention;

FIG. 4 shows heteroduplex and homoduplex fragments;

FIG. 5 shows another embodiment of a methylated cytosine detection device in accordance with the present invention;

FIG. 6 illustrates a temperature-time profile having a three different ramp periods according to the invention;

FIGS. 7a and 7b show fluorescence intensity data of a first unknown and a first reference nucleotide-containing compound, respectively;

FIGS. 7c and 7d show fluorescence intensity data of a second unknown and a second reference nucleotide-containing compound, respectively;

FIG. 8 shows a flow chart including steps for analyzing data indicative of the presence of methylated cytosine.

### DETAILED DESCRIPTION OF THE PREFERRED EMBODIMENT

The present invention relates to a method for obtaining data indicative of the presence of one or more methylated cytosines in a nucleotide-containing compound (NCC), such as a DNA sequence. As used herein, the terms NCC and are used interchangeably in reference to compounds that include at least one sequence of nucleotides. The NCC is preferably contacted with a compound composed to replace non-methylated cytosines of the NCC with another base to provide a modified NCC. The modified NCC is subjected to temperature gradient electrophoresis whereby spectroscopic signals are obtained. The spectroscopic signals are converted to the data indicative of the presence of the one or more methylated cytosines. Preferably, the indicative data can be used to determine whether the nucleotide-containing compound included one or more methylated cytosine.

Sample Modification

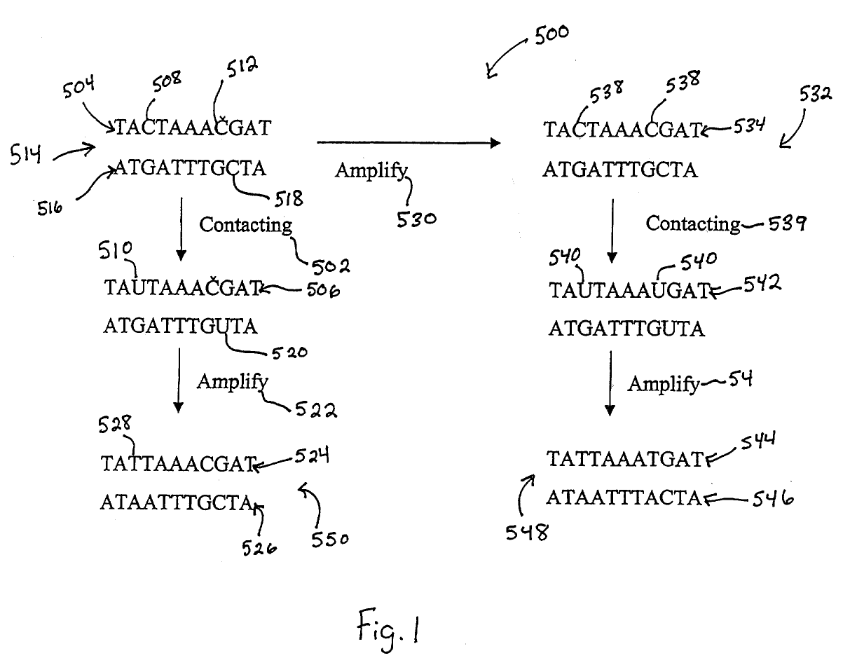

Referring to a flow chart 500 of FIG. 1, a method of the invention includes a contacting step 502, which includes contacting a denatured NCC with a compound suitable to provide a modified NCC 506 in which non-methylated cytosines 508 have been replaced with a different base.

A preferred compound is a bisulfite salt, such as sodium bisulfite NaHSO₃, which replaces non-methylated cytosines of an NCC with uracil 510. U.S. Pat. No. 6,017,704 to Herman et al., which is incorporated herein to the extent necessary to understand the present invention, discloses suitable methylation specific bisulfite chemistry that replaces non-methylated cytosines with uracil. It should be understood, however, that compounds other than bisulfite that similarly modify non-methylated cytosine, but not methylated cytosine can also be used in the method of the invention.

Sodium bisulfite reacts readily with the 5,6-double bond of cytosine, but poorly with methylated cytosine. Cytosine reacts with the bisulfite ion to form a sulfonated cytosine reaction intermediate, which is susceptible to deamination, giving rise to a sulfonated uracil. The sulfonate group can be removed under alkaline conditions, resulting in the formation of uracil. Uracil is recognized as a thymine by Taq polymerase and therefore, upon PCR, the resultant product contains cytosine only at the position where methylated cytosines occur. Thus, following the contacting step, the only remaining cytosines of the NCC are methylated.

The NCC may comprise only a single sequence of nucleotides or can comprise a plurality of sequences of nucleotides, such as a double-strand of DNA 514. A complementary NCC 516 of double-stranded DNA 514 is complementary to NCC 504. Non-methylated cytosines 518 of NCC 516 are preferably replaced with the same base that replaced non-methylated cytosines of NCC 504.

Once non-methylated cytosines of NCC 504 have been replaced with another base, at least a portion of modified NCC 506 is amplified 522 via a polymerase chain reaction (PCR). The amplification is preferably performed in the presence of strand-specific primers, which amplify only modified NCC 506 to provide a PCR product NCC 524. The primers comprise two or more deoxyribonucleotides or ribonucleotides, preferably more than three, and most preferably more than 8, which sequence is capable of initiating synthesis of a complementary primer extension product.

The amplification is preferably performed in the presence of nucleoside triphosphates, an agent for polymerization, such as DNA polymerase, and a suitable temperature and pH.

If the NCC is one strand of a double-stranded NCC, the strand-specific primers amplify only the NCC that is to be analyzed to determine the presence of methylated cytosines. Thus, for example, complementary sequence 516 is preferably not amplified. Using strand-specific primers preferably provides PCR products comprising only PCR product 524 and a complementary PCR product sequence 526. In the annealed state, the PCR product 524 and complementary PCR product sequence 526 form a double stranded nucleotide containing compound (DNSCC) 550.

During amplification 522, the bases that had been replaced during the denature/replace step 502 are preferably replaced with another base. For example, uracil bases 510 of modified NCC 506 are preferably converted to another base, such as thymine 528.

Analysis of Modified Samples

To obtain data indicative of the presence of one or more methylated cytosines in NCC 504, the PCR products obtained by amplifying modified NCC 506 are subjected to temperature gradient electrophoresis (TGE). As discussed below, preferred modes of TGE include heteroduplex analsysis by temperature gradient capillary electrophoresis (HA-TGCE) and single-strand conformation polymorphism analysis by temperature-gradient capillary electrophoresis (SSCP-TGCE). In any case, the electrophoresed modified NCC are preferably irradiated with light to provide spectroscopic signals indicative of the presence of the electrophoresed modified NCC. The spectroscopic signals are converted to data indicative of the presence of the one or more methylated cytosines.

The TGE preferably comprises subjecting a reference nucleotide-containing compound (RNCC) to TGE followed by the acquisition of reference spectroscopic signals from the electrophoresed reference nucleotide-containing compound. The reference spectroscopic signals can be converted to reference data. As discussed below, the reference data enhance the ability of the invention to indicate the presence of methylated cytosines.

The RNCC preferably comprises reference PCR products 532 having a known methylation status. The RNCC can be obtained by subjecting a portion of NCC 504 to PCR amplification 530. During the PCR amplification, methylated cytosines 512 are amplified as are non-methylated cytosines 508. Thus, the reference PCR products 532 preferably include a sequence 534 having the same sequence as NCC 504 with the exception that NCC 504 may contain one or more methylated cytosines, whereas the reference PCR products 532 are essentially free of methylated cytosines.

Reference PCR product 532 is contacted 539 with a compound suitable to replace non-methylated cytosines with another base. Preferably, PCR product 532 is denatured and contacted with bisulfite 536 under conditions suitable to replace non-methylated cytosines 538 with uracil 540, thereby providing a modified reference nucleotide containing compound (MRNCC) 542.

The MRNCC 542 is amplified 545 by PCR to provide a second PCR reference product 544. Amplification 545 is preferably carried out under conditions suitable to replace uracil bases of MRNCC 542 with another base, which is preferably thymine. Amplification step 545 also provides a sequence 546, which is complementary to second PCR reference product 544. In the annealed state, second PCR reference product 544 and sequence 546 form a reference double-stranded nucleotide containing compound (RDSNCC) 548.

Heteroduplex Analysis Temperature Gradient Electrophoresis

According to the HA-TGCE method, DSNCC 550 and RDSNCC 548 are combined in preferably equal proportions to form a mixture. The combined nucleotide containing compounds of the mixture are denatured and re-annealed. The denaturing is preferably accomplished by raising the temperature of the mixture by an amount sufficient to melt the nucleotide containing compounds.

The re-annealed nucleotide containing compounds will comprise heteroduplexes and homoduplexes if there is at least one nucleotide difference between two strands of the DSNCC 550 and RDSNCC 548. A nucleotide difference will occur if NCC 504 includes at least one methylated cytosine. Heteroduplexes, which include a base pair mismatch due to the nucleotide difference, will denature at a lower temperature compared to the corresponding homoduplex, which lacks a base pair mismatch. A denatured heteroduplex will exhibit a different electrophoretic mobility than the corresponding annealed homoduplex. Therefore, subjecting homoduplex and heteroduplex sample components to a temperature gradient during electrophoresis will cause the two components to at least partially separate. Thus, a temperature gradient electropherogram will include additional or broadened peaks if the sample components include both heteroduplex and homoduplex components thereby indicating the presence of a methylated cytosines.

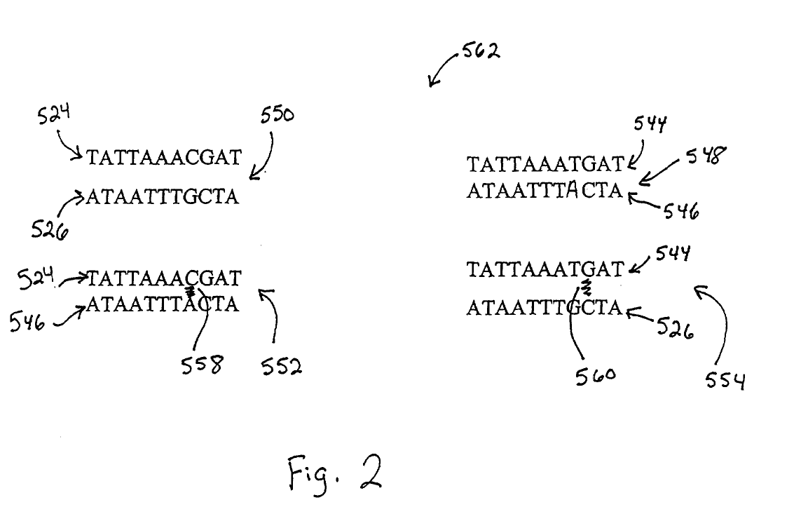

Referring to FIG. 2, the re-annealed nucleotide containing compounds form a mixture 562 containing both homoduplex and heteroduplex components. For example, sequence 524, which is derived from sequence 504 having the methylated cytosine, and complementary sequence 526 may re-anneal to form double stranded nucleotide containing compound 550.

Alternatively, sequence 524 may combine with sequence 546, which is complementary to sequence 544, to form a heteroduplex 552, which includes a base pair mismatch 558. Sequence 544 may recombine with sequence 526, which is complementary to sequence 524, to form a second heteroduplex. Second heteroduplex includes a base pair mismatch 560. An electropherogram obtained by subjecting mixture 562 to temperature gradient electrophoresis will include additional or broadened peaks because of the presence of the both homoduplex and heteroduplex sample components caused by the methylated cytosine in NCC 504. In the absence of the methylated cytosine, the electropherogram would contain fewer or narrower peaks because the heteroduplex sample components would not be present. Thus, the electropherogram includes data indicative of the presence of the methylated cytosine.

Single Strand Conformation Polymorphorism Temperature Gradient Electrophoresis.

The SSCP-TGCE is another temperature gradient electrophoresis method of the invention for obtaining data indicative of the presence of a methylated cytosine. According to the SSCP-TGCE method, amplification steps 522 and 545 are performed in the presence of a pair of fluorescently labeled strand-specific primers. The resulting products are fluorescent and can be detected upon performing temperature gradient electrophoresis. The labeled PCR products are denatured and subjected to capillary electrophoresis using a temperature gradient. By comparing electrophoregram data generated by an unknown sample to an non-methylated mC control or reference sample, the status of methylation will be determined. Pattern differences are caused by the subtle sequence differences between unknown sample and its control after non-methylated cytosine to uracil to thymine conversion. The slightly different sequences of the resulting strands create conformational differences during electrophoresis.

Temperature Gradient Electrophoresis

In the method of the invention, a temporal temperature gradient is applied to a temperature controlled zone of an electrophoretic separation medium. During the temperature gradient, each NCC in the sample preferably experiences a first temperature where the NCC is not melted and a second, higher temperature where the NCC is melted. Of course, the first and second temperatures will likely be different for different NCCs. As used herein, the term melt is synonymous with the term thermally denature. The temperature of the temperature controlled zone preferably changes by at least about 1° C. and more preferably at least about 7.5° C. The temperature of the temperature controlled zone is set with a precision of better than about 0.02° C.

The presence of sample components is determined by obtaining spectroscopic signals indicative of the presence of sample components. The spectroscopic signals can include, for example, absorbance signals or fluorescence signals. The spectroscopic signals are converted to data indicative of the presence of methylated cytosines in the sample NCC.

In one embodiment, the electrophoretic separation medium comprises an intercalating dye, such as ethidium bromide to allow fluorescence detection of the separated NCCs. The intercalating dye preferentially allows detection of double stranded DNA as compared to single stranded DNA. In one embodiment, the separation medium is substantially free of a covalent tag suitable for fluorescence detection of single strands of DNA and the separation medium is completely free of a covalent tag. By substantially free it is meant that the presence of any covalent tag suitable for fluorescence detection of single strands of DNA is insufficient to interfere with the detection of sample compounds using fluorescence resulting from the intercalating dye. In one embodiment, the NCCs to be separated are preferably substantially free of fluorescent dyes that covalently tag single stranded DNA. Multiple samples comprising NCCs, such as DNA fragments, can be simultaneously analyzed.

In another embodiment, the electrophoretic medium comprises a tagging agent, such as an intercalating tag, having an extinction coefficient that is sufficiently large to allow the presence of the sample components to be determined by detecting the absorbance of the tagging agent.

In another embodiment, the presence of the NCCs is determined by directly measuring the absorbance of the sample components themselves rather than by measuring the absorbance of a tagging agent.

The fluorescence intensity data is indicative of the presence of methylated cytosines in the sample NCC. By indicative, it is meant that the fluorescence data of the sample components can be compared with fluorescence data obtained from reference sample components to determine the presence of a methylated cytosine. The presence of methylated cytosines are preferably identified by comparing electrophoretic fluorescence intensity data resulting from a heteroduplex NCC with electrophoretic fluorescence intensity data resulting from a homoduplex reference NCC without prior knowledge of the DNA sequence.

The invention is suitable for high-throughput screening by multiplexing large numbers of samples. Preferably, at least as many as 96 electrophoretic separations can be simultaneously performed.

Temperature Control

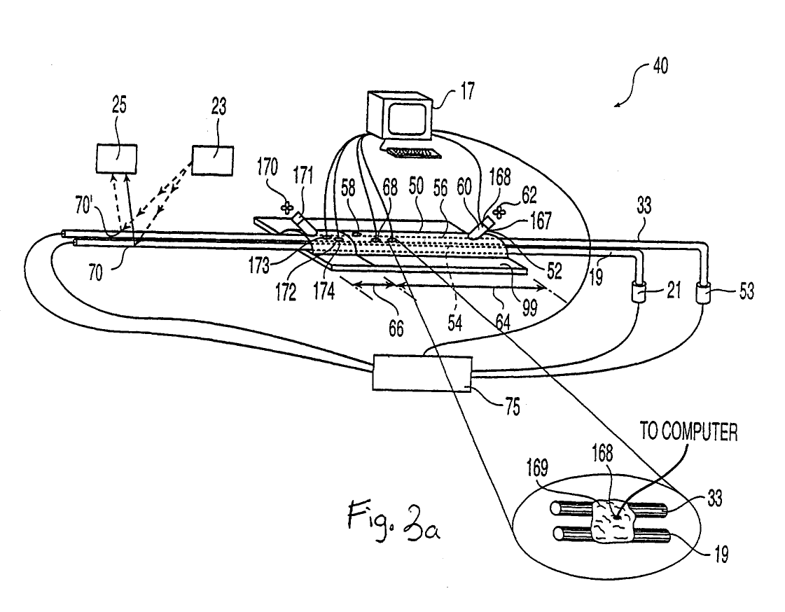

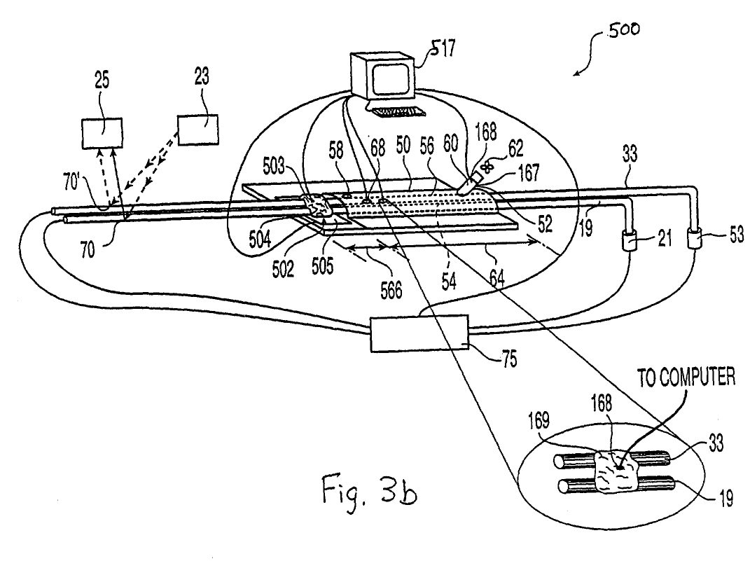

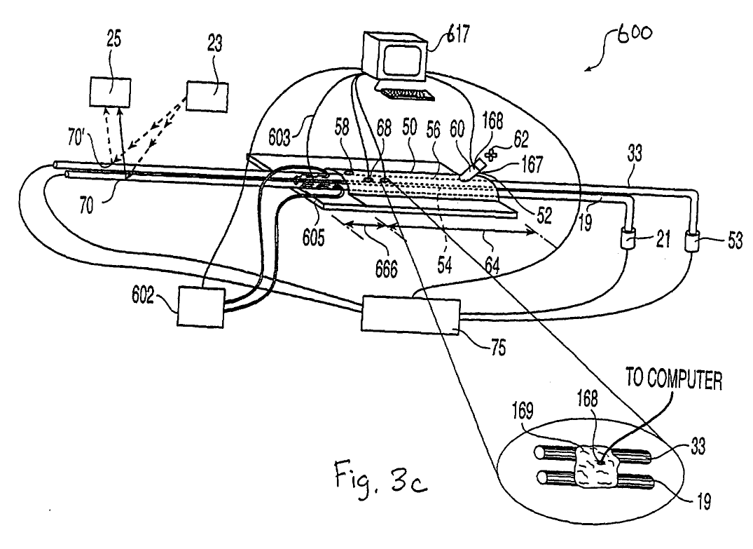

FIG. 3a shows a preferred arrangement of an embodiment of the present electrophoretic methylated cytosine detection device 40. A separation lane, such as a sample capillary 33, is provided to electrophoretically separate unknown sample compounds. By separation lane, it is meant any structure configured and arranged to separate a sample using electrophoresis. Preferred structures include capillaries and microfabricated channels. The separation takes place within the internal bores of the capillaries or the interior of the microfabricated channels. As discussed below, the internal bore or interior of the channels are filled with a separation medium suitable for supporting an electrophoretic separation.

Capillary 33 is arranged to be in fluid contact with a sample reservoir 53, which is configured to contain a volume of sample sufficient to perform an analysis. The sample is preferably suspended or dissolved in a buffer suitable for electrophoresis. Examples of suitable sample reservoirs include the wells of a microtitre plate, a vessel configured to perform PCR amplification of a volume of sample, a reservoir of a microfabricated lab on a chip device, and the like.

Methylated cytosine detection device 40 is preferably provided with an optional reference capillary 19 configured to simultaneously separate a reference sample comprising reference NCCs. Reference capillary 19 includes a reference reservoir 21 configured to contain the reference sample. Reference capillary 19 and reference reservoir 21 have the same characteristics as the sample capillary 33 and sample reservoir 53. An optional support 99 is provided to stabilize capillaries 19, 33.

Device 40 includes a power supply 75 for providing a voltage and current sufficient for electrophoretic separation of a sample. The power supply is preferably configured to allow at least one of the current or resistance of the capillary to be monitored during a separation. Preferably, the current or resistance data is received by the computing device 17 to allow the electric potential to be varied to maintain a constant current or resistance. This is discussed in more detail below.

A temperature control zone 50 of sample capillary 33 and optional reference capillary 19 are placed in thermal contact with an external heat source, such as a gas, which is used to heat portions of capillaries 33, 19. Air or nitrogen are examples of gas that can be used. Because the capillaries 33, 19 preferably have a radius of less than about 500 microns, the thermal conductivity between the separation medium within the internal bores of the capillaries and the gas is sufficiently high to allow the gas to heat the separation medium. Thus, during electrophoresis, the external heat source, rather than ohmic heating of the separation medium itself, is the dominant source of any substantial temperature changes or fluctuations within the separation medium within the capillary. Because sample components, such as NCCs, migrate within the separation medium, which typically contains a liquid, the sample components are also in thermal contact with the external heat source.

Temperature control zone 50 preferably extends for a length Lₜₑₘₚ 64 of the capillaries. At least one inlet port 52 is provided to introduce the heated gas to a heated region 54 between the capillaries and a thermal jacket 56. At least one outlet 58 is provided to allow the gas to exit from heated region 54. A fan 62 or other device to force the gas into the inlet and out of the exit is provided. Thermal jacket 56, which can entirely surround capillaries 33, 19, insulates temperature control zone 50 to reduce heat loss therefrom and to maintain the gas in contact with capillaries 33, 19.

The gas can be heated by, for example, passing the gas over a resistively heated filament 167 or a heat exchanger prior to introducing the gas into heated region 54. Filament 167 can be located within or adjacent inlet port to reduce heat loss that would occur if hot gas were transported from a location remote from device 40.

At least one temperature sensor 68 is preferably used to determine the temperature of the gas in contact with capillaries 33, 19 in the portion Lₜₑₘₚ. An additional temperature sensor 168 is placed in thermal contact with the capillaries in the portion Lₜₑₘₚ. Preferably, sensor 168 is embedded in a mass of thermally conductive material 169, so that the temperature reported by sensor 168 is indicative of the temperature within the internal bore of capillaries 33, 19. Suitable thermally conductive materials include, for example, the TCE series of thermal epoxies available from Melcor, Trenton, N.J.

A computer 17 receives signals from sensors 68, 168 indicative of the gas temperature, and capillary temperature, respectively. The temperature of filament 167 is preferably under control of computer 17, which is configured to vary the current flowing through the filament. During operation, computer 17 compares the temperature received from sensor 168 (capillary bore temperature) with a predetermined target temperature, which can vary as a function of time. If the capillary bore temperature is less than the target temperature, computer 17 raises the temperature of filament 167, such by increasing the amount of current flowing through filament 167, to increase the gas temperature in contact with capillaries 33, 19. Conversely, if the capillary bore temperature is greater than the target temperature, computer 17 lowers the temperature of filament 167, such by decreasing the amount of current. The difference between the temperature received from sensor 68, which measures the gas temperature, and the temperature from sensor 168 is used to determine relative change in filament temperature that is required to reach the target temperature. For example, if the temperatures of the gas and capillary bores are each significantly less than the target temperature, a greater increase in the filament temperature is required than if only the capillary bore temperature is significantly less than the target temperature.

As an alternative to controlling the gas temperature by varying a filament temperature, the gas temperature can be varied by mixing a first hot gas and a second, cooler gas. By varying the ratio of the gas volumes in the mixed stream, the temperature can be varied. A mass flow controller, such as the Type 1179A General Purpose Mass Flow Controller provided by MKS Instruments of Andover, Mass., can be used to obtain and measure a variable degree of mixing between the two gas sources.

Controlling the temperature of the sample components within the capillary by use of a gas rather than by using a liquid, allows the temperature of the capillary bore (and sample components therein) to be changed much more rapidly because the temperature of the gas can be changed much more rapidly than the temperature of a liquid. It should be understood, however, that, where rapid temperature changes are not required, a liquid may be used to control the temperature of the temperature control zone.

A portion L_{cool} 66 of capillaries 33 and 19 can be provided to reduce the temperature of sample components, such as NCCs, after the samples have passed through the temperature control zone. Cooling the sample components can provide an increase in detection efficiency, as discussed below. The temperature in portion L_{cool} 66 can be controlled using chilled gas with an arrangement similar to that provided in the temperature control zone. Because the radial dimensions of capillaries 33, 19 are on the order of about 500 microns or less, cooling the capillaries themselves serves to cool sample components migrating within the separation filling the internal bores of the capillaries. Thus, the chilled gas in the portion L_{cool} is in thermal contact with sample components present within the internal bores of capillaries 33, 19.

A fan 170 or other air circulation device is provided to introduce chilled gas into an inlet port 171. Upon entering the inlet port 171, the chilled gas comes into thermal contact with the portions capillaries 33, 19 disposed in L_{cool} and sample components present in the cooled capillary portions. The chilled gas entering input port 171 can be provided by, for example, contacting the gas with a condenser or heat exchanger filled with a chilled liquid. An outlet port 172 allows chilled gas to escape.

A sensor 173 monitors the gas temperature within L_{cool} 66 and a sensor 174, which is in thermal contact with capillaries 33, 19, determines the temperature within the bores of the capillaries. Computer 17 preferably receives signals from sensors 173, 174. As the temperature within the temperature controlled portion of the system increases, additional cooling may be required to maintain a predetermined target temperature within L_{cool}. If computer 17 determines that the temperature within L_{cool} is greater than the target temperature, the gas flow rate through L_{cool} can be increased, such as be increasing the fan speed.

Device 40 also includes a light source 23, such as a laser emitting a wavelength suitable to generate fluorescence from the intercalating dye. A detector 25 is arranged to obtain fluorescence intensity data, such as a time-intensity electropherogram including peaks indicative of the presence of NCCs, and send the detected fluorescence intensities to computing device 17.

Referring to FIG. 3b, a methylation detection system 500 having a thermoelectric cooler, such as a Peltier cooler 502, to cool samples that have been subjected to temperature gradient electrophoresis is shown. An example of a suitable Peltier cooler is the Thermo-Electric Module No. 01/128/040 available from Ferrotec America Corporation, Nashua, N.H. Peltier cooler 502 cools at least a portion of capillaries 33, 19 disposed in a cooled portion L_{cool} 566. A chilled side 505 of Peltier cooler 502, which is controlled by computer 517, is disposed in thermal contact with a portion of capillaries in L_{cool}. By cooling a portion of capillaries 33, 19, cooler 502 also cools samples within the cooled portions of the capillaries. As understood in the art, Peltier coolers release heat on a side that opposes the chilled side. Thus, device 500 preferably includes an apparatus, such as for circulating water or flowing gas, to remove heat from the Peltier cooler.

Thermal contact between Peltier cooler 502 and capillaries 33, 19 is preferably enhanced by using a thermally conductive material, such as a thermal paste 504, which surrounds a portion of the capillaries in contact with Peltier cooler 502. Computer 517 receives signals from a temperature sensor 503 indicative of the temperature within the internal bore of capillaries 33, 19. Computer 517 can vary the cooling level of Peltier cooler 502 by varying the current supplied to the device, as understood in the art. During operation, computer 517 compares the temperature determined by sensor 503 with a predetermined target temperature and increases or decreases the cooling level of Peltier cooler 502 if the temperature is too high or low, respectively.

Referring to FIG. 3c, a device 600 having a liquid chiller 602, is shown. An example of a suitable chiller is the MLA270 Series chiller available from Melcor, Trenton, N.J. Liquid chiller 602, which is under the control of a computer 617, circulates a chilled liquid, such as water, a poly-alcohol, or mixture thereof, through tubing 605, which is in thermal contact with the portions of capillaries 33, 19 disposed in portion L_{cool} 666. Computer 617 receives signals from a temperature sensor 603 disposed in thermal contact with capillaries 33, 19 in portion L_{cool}. When the temperature indicated by sensor 603 deviates from a predetermined target temperature, computer 617 instructs chiller 602 to decrease or increase the temperature of the liquid flowing through tubing 605 depending upon whether the sensor 603 temperature is too high or low, respectively.

The temperature and length of portions L_{cool} 66, 566, 666, hereinafter referred to collectively as L_{cool}, are preferably low enough and long enough, respectively, to allow DNA fragments that are thermally partially denatured within temperature control zone 50 to anneal prior to being detected at a reference detection zone 70 or a sample detection zone 70′. Because the system preferably uses an intercalating dye that is selective for double stranded DNA fragments, allowing denatured fragments to substantially re-anneal enhances the detection sensitivity. The temperature of L_{cool} is reduced to less than about 35° C., preferably less than about 25° C., more preferably less than about 20° C., and most preferably less than about 15°.

In any embodiment of the present invention, the fluorescence intensity data of the sample is preferably obtained simultaneously with the fluorescence intensity data of the reference sample. By “simultaneously,” it is meant that the unknown and reference samples are elecrophoresed in a total time that is at least about 25% less, preferably about 50% less, than twice the time required to sequentially electrophorese the samples. Preferably, the unknown sample is subjected to capillary electrophoresis in the sample capillary and the reference sample is subjected at substantially the same time to capillary electrophoresis in a second, different capillary.

Sample components, such as first and second pairs of NCCs, can be subjected to temperature gradient electrophoresis in the presence of more than one DNA staining dye. The different intercalating dyes preferably fluoresce at wavelengths that are sufficiently different to allow the presence of one of the dyes to be detected even when the other dye is also present. To simultaneously detect fluorescence from each of two or more dyes, the methylated cytosine detection detector preferably comprises a light dispersing element, such as a grating or prism, and a two-dimensional detector, such as a charge coupled device. An example of a suitable detector is described in U.S. Pat. No. 6,118,127, which is incorporated herein to the extent necessary to understand the present invention.

Each pair of NCCs that are separated in the presence of the two intercalating dyes comprises two member NCCs. Each member nucleotide is preferably a double stranded nucleotide, such as a heteroduplex or homoduplex DNA strand. Preferably, one of the intercalating dyes interacts preferentially with the first pair of NCCs and the second intercalating dye interacts preferentially with the second pair of NCCs. Thus, it is possible to determine the presence of both members of each of the first and second pairs of NCCs even if the pairs do not become spatially resolved during electrophoresis.

Separation Media

A preferred separation medium for methylated cytosine detection comprises a buffer, such as 1×TBE buffer, which can be prepared, for example, by dissolving 8.5 g premixed TBE buffer powder (Amerosco, Solon, Ohio) into 500 ml dionized water. An intercalating dye, such as Ethidium bromide is incorporated into the TBE buffer at a concentration sufficient to provide detection of double stranded DNA in the sample. The suitable dye concentration depends upon the particular sample and can be determined by, for example, varying the dye concentration in a series of standard samples to obtain a calibration curve of intensity versus dye concentration. As an alternative to an intercalating dye, a dye that covalently binds to the DNA can be used. An intercalating dye is preferred, however, at least because the intercalating dye can be added to the running buffer. Thus, a separate step to tag the strands of DNA is not required.

The present invention preferably allows methylation detection of DNA fragments from PCR products without first desalting or substantially purifying the products, such as by a filtration or pre-separation. In particular, the present method can be performed without removing single stranded DNA from the PCR products. This is especially important in methylation detection where the sample contains other biological tissues, cells, or reagents. Sampling PCR reaction products, which may contain single strand sequences of DNA, without first desalting or purifying the products is made possible at least in part by the use of an intercalating dye, which preferably associates selectively with double stranded DNA rather than single stranded DNA. The PCR products would have to be depleted of single stranded DNA if traditional dye labels were used because the fluorescence signals from the labeled single strands would interfere with detection of the desired double stranded fragments.

Additionally, the present methylation detection device is preferably configured to inject a high pressure fluid through each separation capillary to reduce memory effects from previous analyses.

A sieving matrix can be prepared using Polyvinylpyrrolidone (PVP) which is available from Sigma (St. Louis, Mo.). A preferred sieving matrix can be made by dissolving about 0.5% to about 6% (w/v) of 360,000 M PVP into 1×TBE buffer with the intercalating dye. Preferably, the amount of PVP is about 3% (w/v). The viscosity of a three percent solution is less than 10 cp. The use of polyvinylpyrrolidone makes the capillary regeneration process very easy to implement. The capillaries have a negligible failure rate even over several months. The excellent EOF suppressing effect of the PVP medium enhances the reproducibility of decreases uncertainty associated with methylated cytosine detection. Alternatively the separation medium includes other sieving matrices such as polyacrylamide gels.

Generating a Temperature Profile

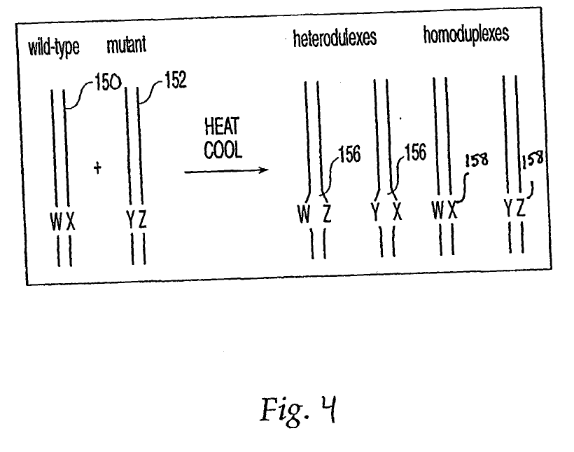

FIG. 4 illustrates the creation of a mixture of heteroduplexes and homoduplexes by combining a first NCC 150 and a reference NCC 152. The letters w, x, y, and z indicate arbitrary nucleotides in the NCCs. The first NCC 150 is preferably derived from a sample NCC by contacting the sample NCC with a bisulfite salt to provide a modified NCC and amplifying the modified NCC, as described above. The reference NCC is preferably obtained by amplifying the sample NCC to provide an amplified NCC. The amplified NCC is contacted with bisulfite to provide a modified amplified NCC, which is then amplified to provide the reference NCC.

The first NCC 150 and reference NCC 152 are combined under conditions suitable to hybridize the NCCs, such as by thermally denaturing and cooling the NCCs. If the sample NCC includes at least one methylated cytosine, the first NCC 150 and reference NCC 152 will have at least one nucleotide difference therebetween. Therefore, hybridized mixture will include heteroduplex NCCs 156 and homoduplex NCCs 158 if the sample NCC contains methylated cytosine, but only homoduplex NCCs if the sample NCC contains no methylated cytosine. Using a temperature profile of the present invention, the presence of heteroduplexes and, hence, methylated cytosines, can be determined.

In a sample containing both a heteroduplex and the corresponding homoduplex, the heteroduplex will melt (denature) at a lower temperature because the heteroduplex contains a base-pair mismatch. Melting occurs because the thermal energy of the separation medium is sufficient to overcome at least some interaction forces between a pair of DNA strands, at least partially denaturing the DNA. When the DNA becomes partially denatured, the mobility of the partially denatured strands decreases in comparison to a pair of equal length strands that are not denatured to the same extent. Therefore, the heteroduplex can be differentiated from the homoduplex by subjecting a sample to separation at a temperature sufficient to melt the heteroduplex but not the homoduplex.

During a separation performed with a ramped temperature profile, the temperature of the separation medium is increased from an initial value that is less than the melting temperature of both the homoduplex and the heteroduplex. As the temperature is raised, the heteroduplex exhibits a retarded migration behavior near its melting temperature compared to the homoduplex. Thus, the two species begin to separate. As the temperature is raised above the melting temperature of the homoduplex, the homoduplex also denatures and the difference in mobilities between the pair of compounds is reduced. Thus, the extent of separation between a homoduplex and heteroduplex depends in part on the total amount of time the separation medium is at a temperature above the melting point of the heteroduplex but less than the melting temperature of the homoduplex. The methylated cytosine can be identified by the difference in the resulting electrophoretic patterns between the homoduplex and the heteroduplex.

A temperature profile of the invention preferably includes at least one change in the temperature of the separation medium as a function of time. Temperatures during the temperature profile can be varied over any time and temperature range sufficient to induce a mobility differential between samples to be separated. In some cases, the analysis objective is to determine if any methylated cytosines are present in a sample and the melting temperatures of any heteroduplex-homoduplex pairs that would indicate presence of a methylated cytosine are not known before the analysis. Here, the temperature is preferably ramped over a wide range that encompasses the melting temperatures of substantially all heteroduplex-homoduplex pairs that might be present in the sample. In other cases, the analysis objective is to determine whether a sample contains a methylated cytosine at a particular location of a NCC. In this situation, the melting temperatures of a heteroduplex-homoduplex pair that would be indicative of the methylated cytosine, if present, are known. As discussed below, the slope of the temperature profile can be optimized to enhance detection of predetermined methylated cytosine.

During electrophoresis, the temperature is preferably above the freezing point of the separation medium, such as above about 0° C., and below the boiling point of the separation medium, such as below about 100° C. The temperature within the temperature control zone is preferably substantially constant along a dimension of the separation medium that is perpendicular to the direction of migration. Thus, for example, the temperature is substantially constant across the radial dimensions of a capillary. By substantially constant temperature it is meant that the spatial temperature variations are insufficient to introduce measurable mobility variations for compounds disposed at different spatial locations within the temperature control zone at any given instant. Thus, at any given instant, the temperature at any point along the portion of each capillary within the temperature control zone is preferably constant, i.e., there are substantially no spatial temperature gradients in the temperature control zone.

For accurate comparison of the patterns, a reproducible temperature profile is required. Because in this invention the temperature of the separation medium can be varied independently of the electric field, arbitrary temperature profiles can be selected without negatively perturbing methylated cytosine detection performance. For example, for the separation of heteroduplex sample compounds using an apparatus and temperature profile of the present invention, migration times have a relative standard deviation of less than 2%.

Because the mobility retardation (differential mobilities between a heteroduplex and corresponding homoduplex) occurs only when the DNA fragments begin to melt, the part of the capillary that is not elevated above the melting temperature of a fragment, will not affect the differential mobility of the fragments. Preferably, a temperature profile of the invention is not begun until at least some and preferably substantially all fragments in a sample have migrated into the temperature control zone.

In order to generate a reasonably accurate range over which to vary the temperature and the rate of temperature variation, the configuration of the capillary layout has to be considered. Preferably, the temperature range and variation rate are appropriate to allow determination of substantially any methylated cytosine in any of the unknown samples being analyzed.

Parameters for a temperature ramping profile preferably include the (1) temperature ramping range from a low temperature T_{L} to a higher temperature T_{H}; (2) time, tᵣ, after injection at which the temperature ramp is initiated; and (3) rate, r, at which the temperature is ramped.

Preferred procedures for determining temperature ramping parameters include (1) selection of the separation voltage and (2) selection of a sample standard that includes DNA fragments covering the size range of fragments in the samples to be analyzed. The voltage depends on the sieving matrix used, the sizes of the fragments to be separated, and the length of the separation lane, as understood in the art.

The sample standard can be a molecular ladder, standards comprising a particular set of fragments, or a combination thereof. The sizes of the fragments range from the smallest fragment F_{S} to the largest fragment F_{L}.

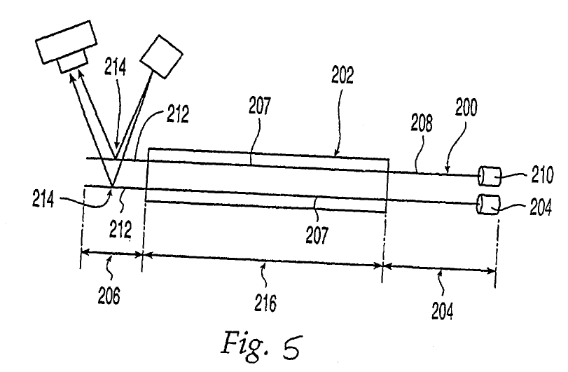

Referring to FIG. 5, an arrangement of multiple capillaries 200 extending through a temperature controlled zone 202 is shown. Capillaries 200 preferably include at least three portions: a first capillary portion 208 preferably extending from a sample injection site 210, a second capillary portion 207 arranged within temperature control zone 202, and a third capillary portion 212 comprising a portion of the capillary between temperature control zone 202 and a detection zone 214. First capillary portion 208 has a length Lᵢₙⱼ 204 between the sample injection site and the temperature control zone. Second capillary portion 204 has a length Lₜₑₘₚ 216 within the temperature control zone. Third capillary portion 212 has a length L_{det} 206 between temperature control zone 202 and detection zone 214.

The internal bores of capillaries 200 preferably comprise a separation medium such as polyvinylpyrolidine to provide separation of DNA fragments. The separation medium preferably contains at least one intercalating dye. An electric field sufficient to electrophoretically separate sample compounds within capillaries 200 is applied at least from sample injection sites 210 to detection zones 214. Sample compounds are preferably introduced (injected) at sample injection sites 210 and migrate under the influence of the electric field through capillary portions 208, 207, and 212, before being detected at detection zone 214. Detection of separated sample compounds is preferably by fluorescence detection of the at least one intercalating dye.

When using the present invention to detect a methylated cytosine, the temperature of the temperature control zone is preferably not modified until all of the species to be separated have entered temperature control zone 202. To determine the time required for all of the sample compounds to enter temperature control zone 202, a standard sample is preferably run first at the temperature Tᵢₙⱼ at which Lᵢₙⱼ will be maintained during the temperature profile. The standard sample preferably comprises fragments having a size range that spans the expected range of fragment sizes in the unknown sample. The migration time, t_{Tinj},F_{L}, for the fragments F_{L} at the large end of the range of fragment size envelop is determined. The largest fragments are typically the slowest moving fragments and have the longest migration times. The migration time is the time required for the sample to migrate from the injection site 210 to the detection zone. Therefore, the time t_{L} required for the largest fragment FL to enter the temperature control zone is given by:

$${t}_{L} = \frac{{L}_{inj}}{{L}_{inj} + {L}_{temp} + {L}_{det}} {t}_{Tinj , FL}$$

After a time t_{L}, the largest (slowest) fragments in the sample will have entered the temperature-controlled zone.

The length of time for the temperature to ramp from the lowest temperature T_{L} to the highest temperature, T_{H}, is also determined. The highest temperature is preferably reached before all of the sample compounds have exited the temperature control zone. The sample standard is run with the temperature control zone set to the highest temperature T_{H}. The migration time t_{TH,FS} for the smallest fragment FS is obtained. The shortest time t_{H} required for the smallest fragment to exit the temperature controlled region with the temperature set at T_{H}, can be estimated as

$${t}_{H} = \frac{{L}_{inj} + {L}_{temp}}{{L}_{inj} + {L}_{temp} + {L}_{det}} {t}_{H , FS}$$

For a temperature profile having a single slope, the temperature ramping rate, r, is given by

$$r = \frac{{T}_{H} - {T}_{L}}{{t}_{H} - {t}_{L}}$$

If the temperature ramping is started right after injection, i.e., before the DNA samples enter the controlled-temperature zone, a ramp beginning at a lower temperature is required to compensate for the temperature ramping that occurs when the sample components are still in the zone of Lᵢₙⱼ. Thus, the starting temperature of the low end of the temperature ramp can be estimated as:

T_{L}=T_{L}−rt_{T}_{_{inj,}}_{FL}

As an example of determining a temperature profile, assume a sample containing DNA fragments ranging from 200 to 500 bp and a capillary having a total length L=Lᵢₙⱼ+Lₜₑₘₚ+L_{det}=4.5 cm+40.5 cm+10.0 cm=55.0 cm

When run at an electrical potential of 10 kV and 35° C. constant temperature, the migration time t_{H,FS} for the 200 bp and 500 bp fragments is about 36 and 55 minutes, respectively. The time t_{L} start the temperature ramping for the controlled-temperature zone can be estimated as:

$${t}_{L} = \frac{{L}_{inj}}{{L}_{inj} + {L}_{temp} + {L}_{det}} {t}_{35 C , FL} = \frac{4.5 cm}{55.0 cm} \times 55 min = 4.5 min$$

When run at 10 kV and 60°, the migration time for the 200-bp DNA fragment is about 27 min. The time for the 200-bp fragment to exit the controlled-temperature zone can then be determined as:

$${t}_{H} = \frac{{L}_{inj} + {L}_{temp}}{{L}_{inj} + {L}_{temp} + {L}_{det}} {t}_{60 , FS} = \frac{45 cm}{55 cm} \times 27 min = 22 min$$

The rate for temperature ramping from 57° to 65° is then estimated as:

$$r = \frac{{T}_{H} - {T}_{L}}{{t}_{H} - {t}_{L}} = \frac{65 ° C . - 57 ° C .}{22 min - 4.5 min} = 0.46 ° C . \text{/} min$$

If the temperature ramp of the temperature control zone is begun when the samples are injected, which is before the samples enter the temperature control zone, the actual starting temperature of the temperature control zone is given by:

$${T}_{L}^{′} = {T}_{L} - {rt}_{L} = 57 ° - \frac{0.46 ° C .}{min} 4.5 min = 55 ° C .$$

Therefore, the temperature ramping profile would be 55° C. to 65° C. over 22 minutes beginning immediately upon the initiation of electrophoresis.

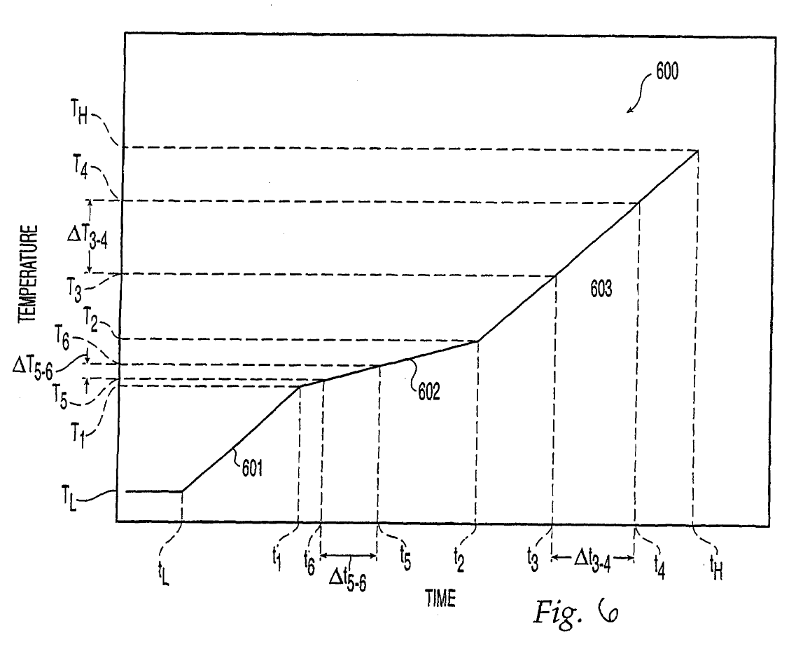

Referring to FIG. 6, a temperature profile 600 having ramp periods with different slopes can provide increased ability to detect methylated cytosines in complex samples. Temperature profile 600 allows the same or better methylated cytosine detection efficiency to be obtained in less than the time required to achieve the same performance using a temperature profile having a single slope. Profile 600 includes 3 temperature ramping periods, although more or fewer ramping periods can be used. Each profile represents the time-changing temperature of sample components present in a temperature control zone.

During a first ramp 601, the temperature of sample components present in the temperature control zone increases from a temperature T_{L} to a temperature T₁. Ramp 601 lasts from a time t_{L} to a time t₁. During a second ramp 602, the temperature of sample components present in the temperature control zone increases with a smaller slope from temperature T₁ to a temperature T₂. Ramp 602 lasts from time t₁ to a time t₂. During a third ramp 603, the temperature of sample components present in the temperature control zone increases from temperature T₂ to a temperature T_{H}. Ramp 603 lasts from a time t₂ to a time t_{H}.

To illustrate how temperature profile 600 improves methylated cytosine detection performance over a single slope profile, consider a sample having a first heteroduplex-homoduplex pair comprising a first heteroduplex that melts at a temperature T₃ and a first homoduplex that melts at a higher temperature T₄ and a second heteroduplex-homoduplex pair comprising a second heteroduplex that melts at a temperature T₅ and a second homoduplex that melts at a higher temperature T₆. Recall that a heteroduplex-homoduplex pair will exhibit different mobilities if the extent of denaturation (melting) of the members of the pair are different.

During a separation, the first pair will exhibit different mobilities between time t₃, when the temperature is T₃, and a time t₄, when the temperature is T₄. Because the temperature melting point differential ΔT₄₋₃=|T₄−T₃| of the first pair is large compared to the range of ramp 600, the first pair exhibits different separation mobilities over a time differential Δt₄₋₃=|t₄−t₃|, which is large compared to the length of ramp 600. The ΔT's are expressed in terms of absolute value because temperature ramps having negative slopes can be used as an alternative to temperature ramps having positive slopes. Therefore, peaks indicative of the presence of the first heteroduplex and first homoduplex should be well resolved and the presence of the corresponding methylated cytosine will not be missed.

The melting point temperature differential ΔT₅₋₆=|T₆−T₅| of the second pair, however, is much less than the melting point temperature differential ΔT₄₋₃ of the first pair. Thus, if the slope of ramp 602 were as large as the slope of ramp 603, the second pair would exhibit differential mobilities only over a narrow range of time and might not be resolved. In FIG. 6, however, ramp 602 has a smaller slope than ramp 603, which compensates for the smaller melting point differential of the second pair. Thus, the second pair exhibits differential mobilities over a time differential Δt₅₋₆, which is sufficiently large to obtain resolution of the member strands of the second pair.

Using multiple slope profile 600 reduces analysis time because if the entire profile had the same smaller slope as ramp 602 a longer period of time would be required to cover the entire temperature range between T_{L} and T_{H}.

A multiple slope profile, such as profile 600, can also improve analysis in other situations. For example, if a sample includes a plurality of fragments that are closely spaced in size so that they exhibit similar mobilities, the slope of the temperature profile can be decreased over a temperature range corresponding to the melting temperatures of the closely spaced fragments. Because of the decreased slope, each heteroduplex and its corresponding homoduplex in the sample will be exposed to a temperature sufficient to melt the heteroduplex but not the homoduplex for a longer period of time. The heteroduplex and homoduplex experience a differential mobility for a longer period of time.

The times at which to initiate and end a given ramp can be determined in several ways. For example, in many methylated cytosine analyses, the melting temperatures of target species, such as a heteroduplex-homoduplex pair, in the sample is known before the analysis is performed. In repetitive analyses, such as clinical assays, the presence of particular target species or the presence of a plurality of closely spaced fragments may also be known prior to the analysis. In these situations, the lower temperature of the ramping period having the lower slope should be lower than the melting temperature of the heteroduplex and the upper temperature of the lower slope ramping period should be higher than the homoduplex.

In one embodiment of the multiple slope temperature ramp, the sample components are subjected to the multiple slope temperature profile during a single electrophoresis run. By electrophoresis run, it is meant an electrokinetic separation that includes the injection, separation, and detection of sample components. Thus, substantially all of the sample components experience both the lower slope temperature ramp and the higher slope temperature ramp. In a different embodiment, the sample components are subjected to temperature gradient electrophoresis, wherein the temperature is changed at a first rate during a first electrophoresis run. During a second electrophoresis run, the sample components are subjected to temperature gradient electrophoresis wherein the temperature is changed at a second, different rate. The first and second electrophoresis runs may be performed sequentially in the same separation lane, such as a capillary or microchannel, or simultaneously in different capillaries or microchannels.

The temperature profile does not have to begin at a lower temperature and increase to a higher value. In one embodiment, a ramp, either linear or non-linear, has a negative slope beginning at a higher temperature and decreasing to a lower temperature while the sample compounds are present in the temperature control zone.

Additionally, more than one temperature profile can be run while a set of sample compounds are present in the temperature control zone. For example, rather than using a single temperature profile that ramps from 60 to 70° C., a set of N temperature ramps can be performed. Preferably, each of the N temperature ramps would range from 60 to 70° C. and back to 60° C. Compared to a single temperature ramp that lasts for a time ts, each of the N temperature ramps would preferably last for a time ts/N. Therefore, if the time ts is less than the time for a given heteroduplex/homoduplex pair to migrate through the temperature control zone, the pair would experience a differential mobility for the same length of time. Each heteroduplex/homoduplex pair comprises two member NCCs, preferably a heteroduplex double strand of DNA and a homoduplex double strand of DNA.

When different portions of a capillary are at different temperatures, the voltage drop along the capillary is not uniform. Therefore, an electric field correction is preferably made to maintain constant mobilities in the portions Lᵢₙⱼ and L_{det}. This correction increases the precision of the observed migration times. Because the conductivity of the capillary portions outside the temperature control zone is independent of temperature within the temperature control zone, the electric field across the capillary should be proportional to the current through the capillary. When performing a temperature profile, the current across the capillary is preferably maintained at the same amperage as the current that was used in running the standard samples as described above. By adjusting the current across the capillary to have the same amperage during the temperature profile, the DNA mobility outside Lₜₑₘₚ should be the same regardless of the temperature of Lₜₑₘₚ. A similar correction could be obtained by maintaining a constant resistance across the capillary during a temperature profile.

It should be emphasized that temperature profiles suitable for use with the methylated cytosine detection device do not have to be a linear function of time but may also be non-linear or include a combination of profile segments that each have a same or different temperature gradient and duration.

Data Indicative of the Presence of Methylated Cytosines

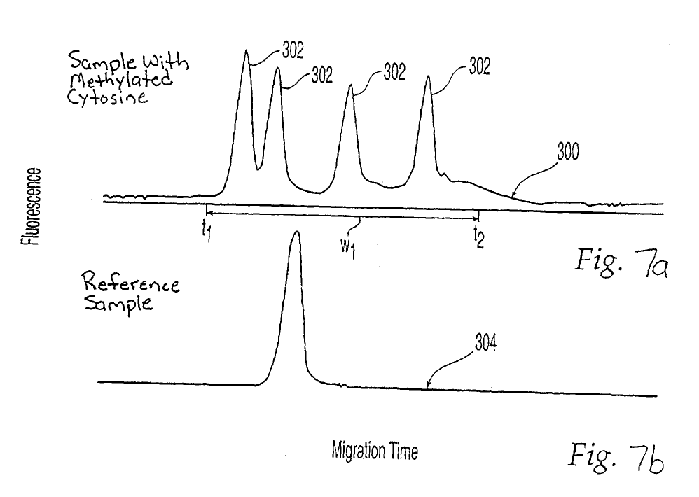

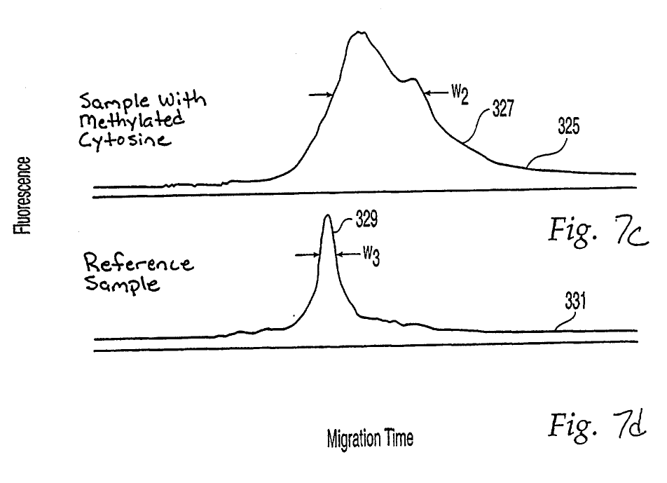

FIGS. 7a and 7b show the fluorescence-migration time data (electropherograms) of two homoduplex samples and the corresponding heteroduplex samples. In these examples, the heteroduplex samples represent samples comprising NCCs having at least one methylated cytosine. As used herein, the term unknown sample indicates a sample that is to be analyzed to determine or confirm the presence of a methylated cytosine in the sample. The homoduplex samples serve as reference samples, which are preferably essentially free of methylated cytosine. Upon comparing the spectroscopic signals or data derived from the spectroscopic signals obtained from the unknown sample with that of the reference sample, it is possible to determine or confirm the presence of methylated cytosine in the unknown sample.

It should be understood that the reference sample does not have to be electrophoresed simultaneously with the unknown sample. Indeed, the spectroscopic data of the unknown sample can be compared with stored reference data, such as data present in a look-up table or other database. For example, the stored reference data can comprise spectroscopic data derived from one or more reference samples that had been previously subjected to temperature gradient electrophoresis.

Referring to FIGS. 7a and 7b, fluorescence intensity data 300 of an unknown sample includes multiple peaks 302 that do not appear in the fluorescence intensity data of the homoduplex reference sample 304. Extra peaks 302 appear within a migration time t₁ and a migration time t₂. The time between migration time t₁ and migration time t₂ is a migration time window w₁.

Referring to FIGS. 7c and 7d, even a slight change in the pattern of peaks is sufficient to indicate the presence of a methylated cytosine in the unknown sample since the present invention provides a highly reproducible system. Perfect separation of the fragments in the heteroduplex samples is not necessary to identify the presence of a methylated cytosine. For example, the presence of a methylated cytosine in the fluorescence intensity data 325 of the unknown NCCs shown in FIG. 7c is evident upon comparing data 325 to the reference data 331 even though a peak 327 of the data 325 is not clearly resolved into its 4 components. In this case, the presence of a methylated cytosine is determined because peak 327 has a width w₂ that is much broader that a width w₃ of a peak 329 observed in the fluorescence intensity data 331 of the reference sample seen in FIG. 7d, which is free of a methylated cytosine. The peak widths are preferably determined at 50% half-maximum intensity, as understood in the art.

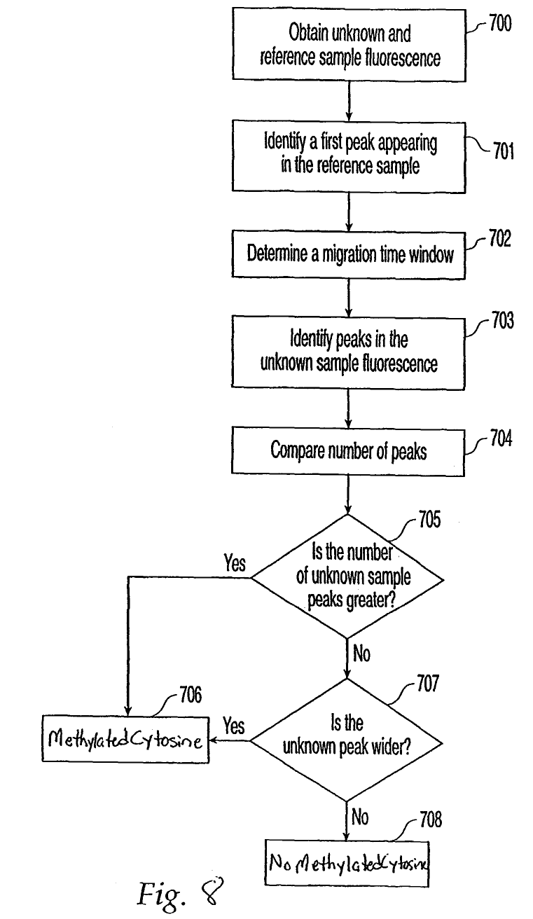

Referring to a flow chart 609 shown in FIG. 8, one embodiment of methylated cytosine detection comprises comparing a first parameter representative of spectroscopic data resulting from an unknown sample with a second parameter representative of a spectroscopic data resulting from a reference sample. For example, the number of peaks appearing in the fluorescence data of an unknown sample can be compared with the number of peaks appearing in the fluorescence data of a reference sample. Flow chart 609 is followed when the reference sample comprises a homoduplex nucleotide containing compound. The methylated cytosine detection process begins by obtaining unknown and reference sample fluorescence 700. The methylated cytosine detection process further involves analysis of the fluorescence data, which analysis is preferably automated and performed by computer, which preferably includes software or a processor programmed to perform the detection process.

The automated comparison process includes identifying 701 a first peak in the reference sample. Peaks can be identified by, for example, establishing an intensity threshold that is greater than the average intensity in the electropherogram. Fluorescence data that have an intensity greater than the threshold intensity are identified as peaks.

A migration time window having a predetermined width is selected 702. The migration time window width is about 15%, preferably about 10% of the migration time of peak identified in the homoduplex fluorescence data. The migration time window is preferably centered about the peak in the homoduplex fluorescence data.

The number of peaks appearing within the migration time window of the fluorescence data of the unknown sample is determined 703 and compared 704 to the number of peaks in the migration time window of the reference sample fluorescence. Typically, there is only one peak in the reference sample migration time window. If the number of peaks in the migration time window of the unknown sample fluorescence exceeds the number of peaks in the migration time window of the reference sample, the presence of methylated cytosine is indicated 706.

If the number of peaks in the unknown sample fluorescence is not greater, the widths of the peaks are determined 707, as discussed above. If the width of the peaks in the unknown sample fluorescence exceeds the width of the corresponding peak in the reference sample fluorescence, the presence of methylated cytosine is indicated. If the widths of the peaks in the fluorescence of the unknown sample and the reference sample are the same 708, the absence of methylated cytosine is indicated.

When the number of peaks in the unknown sample fluorescence exceeds the number of peaks in the reference sample fluorescence, the presence of methylated cytosine is indicated with high confidence. A determination based upon peak width provides lesser assurance. However, a false positive is less of a concern than a false negative in clinical diagnosis, since further tests (such as sequencing) will be performed in these situations. The actual confidence level can be determined from the 2% Relative Standard Deviation (RSD) for the migration times and the level of the pattern change derived from curve fitting. Obviously, if one obtains a negative result in determining the presence of a methylated cytosine in an unknown sample, then the absence of a methylated cytosine in the unknown sample has been determined.

The method illustrated in flow chart 609 can be adapted for analyses performed using a reference sample that contains one or more methylated cytosine. Steps 700, 701, 702, 703, and 704 would be performed as described above. Steps 705, 706, 707, and 708 would be replaced by complementary steps that take account of the fact that, in this adapted method, the reference data would contain a plurality of peaks or a wide peak corresponding to the methylated cytosine.

While the above invention has been described with reference to certain preferred embodiments, it should be kept in mind that the scope of the present invention is not limited to these. Thus, one skilled in the art may find variations of these preferred embodiments which, nevertheless, fall within the spirit of the present invention, whose scope is defined by the claims set forth below.

What is claimed is:

1. A temperature gradient electrophoresis-based method for generating data indicative of the presence of one or more methylated cytosines in a sample comprising a first nucleotide containing compound (NCC) having non-methylated cytosines, comprising:
contacting a first portion of the sample comprising the first NCC with a first compound to thereby provide a modified NCC, wherein non-methylated cytosines of the first NCC are replaced with a different base;
amplifying the modified NCC to obtain first PCR products;
preparing, from a second portion of the sample comprising the first NCC, a reference NCC having a sequence that differs from a sequence of the first PCR products at locations corresponding to the presence of the methylated cytosines in the first NCC;
subjecting the first PCR products to temperature gradient electrophoresis in the presence of the reference NCC;
irradiating the first PCR products and reference NCC with light to thereby generate a spectroscopic signal; and
converting the spectroscopic signal into data indicative of the presence of the one or more methylated cytosines in the first NCC.

2. The method of claim 1, wherein the step of preparing a reference NCC comprises:
amplifying the second amount of the first NCC to prepare second PCR products; and
contacting the second PCR products with a first compound to thereby provide a second modified NCC, wherein non-methylated cytosines of the second PCR products are replaced with a different base.

3. The method of claim 1, wherein uracil is replaced by thymine during the step of amplifying the modified NCC.

4. The method of claim 1, wherein, prior to the irradiating step, the first PCR products and the reference NCC are combined to obtain at least one heteroduplex and at least one homoduplex.

5. The method of claim 1, wherein the amplifying step comprises contacting the first NCC with strand specific primers.

6. The method of claim 1, wherein the strand specific primers comprise a fluorescent tagging compound.

7. The method of claim 1, wherein the first compound comprises a bisulfite salt.

8. The method of claim 1, wherein the different base is uracil.

9. The method of claim 1, wherein the spectroscopic signals are fluorescence signals.

10. The method of claim 1, wherein the spectroscopic signals are absorbance signals.

11. A temperature gradient electrophoresis-based method for generating data indicative of the presence of one or more methylated cytosines in a sample comprising a first nucleotide containing compound (NCC), comprising:
obtaining first PCR products formed by: (a1) contacting the first NCC with a first compound to thereby provide a modified NCC in which non-methylated cytosines are replaced with a different base, and (a2) amplifying the modified NCC to obtain said first PCR products;
obtaining a reference NCC, the reference NCC being prepared from a second portion of the sample comprising the first NCC, the reference NCC having a sequence that differs from a sequence of the first PCR products at locations corresponding to the presence of the methylated cytosines in the first NCC;
subjecting the first PCR products and a reference NCC to temperature gradient electrophoresis,
irradiating the first PCR products and reference NCC with light to thereby generate a spectroscopic signal; and
converting the spectroscopic signal into data indicative of the presence of the one or more methylated cytosines in the first NCC.

12. The method of claim 11, wherein preparing the the reference NCC comprises:
amplifying the second amount of the first NCC to prepare second PCR products; and
contacting the second PCR products with a first compound to thereby provide a second modified NCC, wherein non-methylated cytosines of the second PCR products are replaced with a different base.

13. The method of claim 11, wherein uracil is replaced by thymine during the step of amplifying the modified NCC.

14. The method of claim 11, wherein, prior to the irradiating step, the first PCR products and the reference NCC are combined to obtain at least one heteroduplex and at least one homoduplex.

15. The method of claim 11, wherein the amplifying step comprises contacting the first NCC with strand specific primers.

16. The method of claim 15, wherein the strand specific primers comprise a fluorescent tagging compound.

17. The method of claim 11, wherein the first compound comprises a bisulfite salt.

18. The method of claim 11, wherein the different base is uracil.

19. The method of claim 11, wherein the spectroscopic signals are fluorescence signals.

20. The method of claim 11, wherein the spectroscopic signals are absorbance signals.
